(19) United States
(12) Plant Patent Application Publication
(10) Pub. No.: US 2005/0283876 P1
(43) Pub. Date: Dec. 22, 2005

(54) Campanula plant named 'Royal Wave'

(50) CAMPANULA PORTENSCHLAGIANA; BELLFLOWER

(75) Inventor: Harini Korlipara, Canby, OR (US)

Correspondence Address: KLARQUIST SPARKMAN, LLP, 121 SW SALMON STREET, SUITE 1600, PORTLAND, OR, 97204, US

(73) Assignee: Terra Nova Nurseries, Inc.

(21) Appl. No.: 10/871,832

(22) Filed: Jun. 18, 2004

(51) Int. Cl. A01H 1/00; A01H 5/00; C12N 15/82; C12N 15/87

(52) U.S. Cl. PLT/263

(57) ABSTRACT

A new and distinct hybrid of Campanula plant characterized by large cups of blue purple flowers with light centers and a mounding habit.

Botanical Classification: Campanula hybrid (probably Campanula×pseudoranieri×Campanula carpatica)

Variety Denomination: ‘Royal Wave’

The present invention relates to a new and distinct cultivar of Campanula plant and given the cultivar name of ‘Royal Wave’. Campanula is in the family Campanulaceae. Campanula ‘Royal Wave’ is a tetraploid of Campanula ‘Samantha’, an unpatented, sterile hybrid whose parents probably were C. pseudoranieri×C. carpatica. The tetraploidy was induced under laboratory conditions using colchicine on Stage II plants in tissue culture. Subsequent offspring were trialed and Campanula ‘Royal Wave’ was selected from these.

This new and unique Campanula is distinguished by:

1. Large, wavy, blue purple flowers with light centers.

2. Round habit.

3. Pollen and seed sterility resulting in long bloom time.

This new cultivar has been reproduced only by asexual propagation (tissue culture). Each of the progeny exhibits identical characteristics to the original plant. Asexual propagation by division and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

FIG. 1 shows Campanula ‘Royal Wave’ growing in the ground in the field in July in Canby, Oreg.

FIG. 2 shows a comparison of the flowers of Campanula ‘Samantha’ and Campanula ‘Royal Wave’, with Campanula ‘Royal Wave’ being the flower on the right.

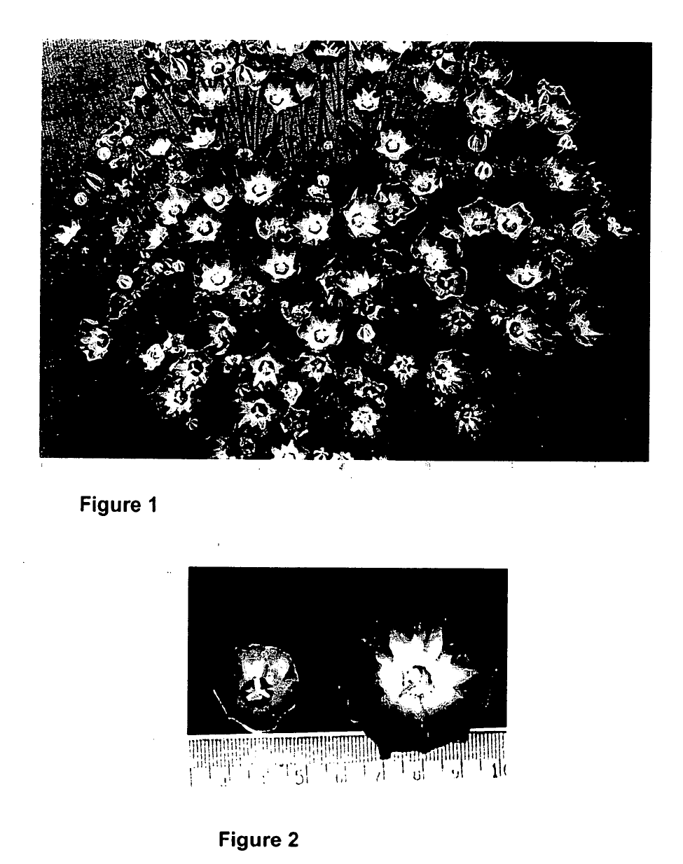

### DETAILED PLANT DESCRIPTION

The following is a detailed description of the new Campanula hybrid based on observations of one and a half year old specimen grown in a one gallon container in a shade house in Canby, Oreg. The color descriptions are all based on The Royal Horticultural Society Colour Chart.
Botanical classification: Campanula hybrid (probably Campanula×pseudoranieri×Campanula carpatica).
Variety denomination: ‘Royal Wave’.
Plant:
   Form.—Tuft, herbaceous perennial.
   Hardiness.—USDA Zone 5 to 9.
   Size.—23 cm tall and 35 cm wide.
   Habit.—Compact, rounded.
Stem:
   Number.—8 to 10, branching at each node, 8 main branches per stem, each of these branching about 4 times.
   Size.—14 to 17 cm long and up to 4 mm wide.
   Color.—Yellow Green 146B.
   Vestiture.—Hispid.
Leaf:
   Type.—Simple.
   Arrangement.—Alternate.
   Shape.—Varies on placement. Lowest leaves on branches are cordate. Most leaves are deltoid. A few leaves at the very ends of the branches are lanceolate.
   Venation.—Pinnate.
   Margins.—Serrate, undulate.
   Apex.—Acuminate.
   Base.—Truncate, sometimes cordate and/or oblique.
   Blade size.—Variable. 8 mm to 2.4 cm long and 5 mm to 2.5 cm wide.
   Vestiture.—Sparsely hispid on margins and bottomside.
   Surface.—Matte.
   Texture.—Thin, leathery.
   Petiole length.—Changes from 3.5 cm at the bottom of the stem to 3 mm at the top.
   Petiole vestiture.—Sparsely hispid.
   Petiole color.—Green 138B.
   Leaf color.—Topside — Green 137A only darker Bottom side — Closest to Green 137C.
Inflorescence:
   Type.—Solitary and terminal.
   Pedicel.—Size — 5 to 10 cm long and 1.5 mm wide Vestiture — Glabrous Color — Green 137A.
   Bloom period.—July through August in Canby, Oreg.
Flower:
   Type.—Actinomorphic.
   Number of flowers.—about 230 per plant.
   Shape.—Broadly campanulate.
   Size.—0.6 to 1 cm deep and 2.5 to 4 cm wide.
   Number of corolla lobes.—5.
   Color.—Violet Blue 93B with lighter centers Violet Blue, 91C.
   Calyx.—Rotate in shape, 5 lobes.
   Calyx size.—1.7 cm wide and deep.
   Calyx lobes.—7 mm long, with 4 to 8 teeth on the lobe margin and tip, cupped, tips acuminate.
   Calyx color.—Green 137B to C.
   Stamen number.—5.
   Pistil color.—Yellow Green 145D.
   Pollen color.—No pollen, male sterile.
   Fragrance.—Sweet.
   Lastingness.—Each flower lasts for about 5 days on the plant.
Fruit and seed: None produced; plant is male and female sterile
Diseases and pests: No diseases found of this new variety or on its parent plant. All Campanula are susceptible to snails and slugs and aphids.

### COMPARISON TO SIMILAR CAMPANULA

Compared to Campanula ‘Samantha’ (an unpatented plant), Campanula ‘Royal Wave’ has larger, thicker, darker flowers with a lighter center. The habit is mounding only rather than mounding to weaving.

Compared to Campanula carpatica and its cultivars with blue purple flowers, Campanula ‘Royal Wave’ is a mounding-clumper rather than a slow creeper, with larger, thicker, and darker flowers. They have similar leaves in their margins and shape. The new variety has larger leaves.

1. A new and distinct Campanula plant substantially as shown and described, characterized by large cups of blue purple flowers with light centers and a mounding habit.
